(19) United States
(12) Patent Application Publication
(10) Pub. No.: US 2023/0027335 A1
(43) Pub. Date: Jan. 26, 2023

(54) IMAGE PROCESSING DEVICE, IMAGE PROCESSING SYSTEM, IMAGE DISPLAY METHOD, AND IMAGE PROCESSING PROGRAM

(71) Applicants: TERUMO KABUSHIKI KAISHA, Tokyo (JP); Rokken Inc., Osaka (JP)

(72) Inventors: Yasukazu SAKAMOTO, Hiratsuka-shi (JP); Katsuhiko SHIMIZU, Fujinomiya-shi (JP); Hiroyuki ISHIHARA, Tokyo (JP); Clément JACQUET, Osaka (JP); Stephen TCHEN, Osaka (JP); Thomas HENN, Osaka (JP); Ryosuke SAGA, Osaka (JP)

(73) Assignees: TERUMO KABUSHIKI KAISHA, Tokyo (JP); Rokken Inc., Osaka (JP)

(21) Appl. No.: 17/957,318

(22) Filed: Sep. 30, 2022

Related U.S. Application Data
(63) Continuation of application No. PCT/JP2021/011535, filed on Mar. 19, 2021.

(30) Foreign Application Priority Data
Mar. 31, 2020 (JP) 2020-061802

(51) Int. Cl. G06T 15/08; G06V 10/56; G06T 7/60; G06T 7/90; G06F 3/14

(52) U.S. Cl. CPC G06T 15/08; G06V 10/56; G06T 7/60; G06T 7/90; G06F 3/14; G06T 2207/10024; G06T 2207/10072

(57) ABSTRACT

An image processing device is an image processing device configured to cause a display to display three-dimensional data as a three-dimensional image, the three-dimensional data representing a biological tissue. The image processing device includes: a control unit configured to adjust a color tone of each pixel of the three-dimensional image according to a dimension of the biological tissue in a linear direction from a viewpoint when the three-dimensional image is displayed on the display.

### CROSS-REFERENCES TO RELATED APPLICATIONS

This application is a continuation of International Application No. PCT/JP2021/011535 filed on Mar. 19, 2021, which claims priority to Japanese Patent Application Serial No. 2020-061802 filed on Mar. 31, 2020, the entire content of both of which is incorporated herein by reference.

### TECHNOLOGICAL FIELD

The present disclosure relates to an image processing device, an image processing system, an image display method, and an image processing program.

### BACKGROUND DISCUSSION

U.S. Pat. No. 8,077,947 discloses a technique of displaying a three-dimensional image in which an endocardial surface is color-coded such that a region in which a myocardium is relatively thick is blue and a region in which a myocardium is relatively thin is red.

Treatment using intravascular ultrasound (IVUS) is widely executed for a cardiac cavity, a cardiac blood vessel, a lower limb artery region, and the like. IVUS is a device or a method for providing a two-dimensional image of a plane perpendicular to a long axis of a catheter.

At present, an operator needs to execute treatment while reconstructing a three-dimensional structure by stacking the two-dimensional images of IVUS in his/her head, which is a barrier particularly to young doctors or inexperienced doctors. In order to remove such a barrier, it is conceivable to automatically generate a three-dimensional image expressing a structure of a biological tissue such as the cardiac cavity or the blood vessel from the two-dimensional images of IVUS and display the generated three-dimensional image toward the operator. When displaying the three-dimensional image, it is conceivable to color-code a tissue surface according to a thickness of the biological tissue. Here, the “thickness” refers to a minimum distance from one tissue surface at any position of the biological tissue to the other tissue surface through the tissue at the position, and is also referred to as a dimension in a thickness direction.

When the surface of the biological tissue has a concave-convex structure, if the tissue surface is color-coded according to the dimension in the thickness direction, the concave-convex structure may be difficult to understand when the operator views the surface of the biological tissue straight in the three-dimensional image. For example, assuming that there is a convex portion that protrudes in a direction intersecting a visual line direction and has a small dimension in the thickness direction, the convex portion is projected as a thin tissue on the tissue surface positioned behind the convex portion as viewed from the operator. As a result, the operator may mistakenly recognize that the tissue behind the convex portion is relatively thin, instead of the convex portion.

### SUMMARY

The present disclosure helps facilitate an understanding of a structure of at least a part of a biological tissue in a visual line direction when a three-dimensional image is displayed.

An image processing device according to an aspect of the present disclosure is an image processing device configured to cause a display to display three-dimensional data as a three-dimensional image, the three-dimensional data representing a biological tissue. The image processing device includes: a control unit configured to adjust a color tone of each pixel of the three-dimensional image according to a dimension of the biological tissue in a linear direction from a viewpoint when the three-dimensional image is displayed on the display.

In one embodiment, the control unit is configured to set a color of a pixel among a pixel group of the three-dimensional image in which the dimension of the biological tissue in the linear direction is smaller than a first threshold value to a first color different from those of the other pixels.

In one embodiment, the control unit is configured to switch a display mode between a first mode of adjusting the color tone of each pixel of the three-dimensional image according to the dimension of the biological tissue in the linear direction, and a second mode of adjusting the color tone of each pixel of the three-dimensional image according to a dimension of the biological tissue in a thickness direction of the biological tissue.

In one embodiment, in the second mode, the control unit sets a color of a pixel among a pixel group of the three-dimensional image in which the dimension of the biological tissue in the thickness direction of the biological tissue is larger than a second threshold value to a second color different from those of the other pixels.

In one embodiment, in the second mode, the control unit sets, among a pixel group of the three-dimensional image, a color of a pixel in which the dimension of the biological tissue in the thickness direction of the biological tissue is larger than a second threshold value to a second color, sets a color of a pixel in which the dimension of the biological tissue in the thickness direction of the biological tissue is smaller than a third threshold value that is less than the second threshold value to a third color different from the second color, and sets a color of a pixel in which the dimension of the biological tissue in the thickness direction of the biological tissue is equal to or greater than the third threshold value and equal to or smaller than the second threshold value to a color different from the second color and the third color.

In one embodiment, the control unit is configured to form, in the three-dimensional data, an opening for exposing a lumen of the biological tissue in the three-dimensional image, and to adjust a position of the viewpoint according to a position of the formed opening.

An image processing system according to an aspect of the present disclosure includes: a sensor configured to acquire tomographic data of the biological tissue while moving in a lumen of the biological tissue; and the image processing device configured to generate the three-dimensional data based on the tomographic data acquired by the sensor.

In one embodiment, the image processing system further includes: the display.

An image display method according to an aspect of the present disclosure is an image display method for causing a display to display three-dimensional data as a three-dimensional image, the three-dimensional data representing a biological tissue. The image display method includes adjusting, by a processor, a color tone of each pixel of the three-dimensional image according to a dimension of the biological tissue in a linear direction from a viewpoint when the three-dimensional image is displayed on the display.

A non-transitory computer-readable medium (CRM) storing computer program code executed by a computer processor that executes an imaging process comprising: displaying, on a display, three-dimensional data as a three-dimensional image, the three-dimensional data representing a biological tissue; and adjusting a color tone of each pixel of the three-dimensional image according to a dimension of the biological tissue in a linear direction from a viewpoint when the three-dimensional image is displayed on the display.

According to the present disclosure, it is possible to facilitate understanding of a structure of at least a part of a biological tissue in a visual line direction when a three-dimensional image is displayed.

### BRIEF DESCRIPTION OF THE DRAWINGS

FIG. 1 is a perspective view of an image processing system according to an aspect of the present disclosure.

FIG. 2 is a perspective view of a probe and a drive unit of the image processing system according to the aspect of the present disclosure.

FIG. 3 is a block diagram illustrating a configuration of an image processing device according to the aspect of the present disclosure.

FIG. 4A is a transverse cross-sectional view illustrating a ridge dimension in a visual line direction and a ridge dimension in a thickness direction.

FIG. 4B is a vertical cross-sectional view illustrating a color tone change based on a dimension in the thickness direction.

FIG. 4C is a vertical cross-sectional view illustrating a color tone change based on a dimension in the visual line direction.

FIG. 5 is a flowchart illustrating an operation of the image processing system according to the aspect of the present disclosure.

FIG. 6 is a diagram illustrating a positional relation among a cross section of a biological tissue, an opening, and a viewpoint in the aspect of the present disclosure.

FIG. 7 is a diagram illustrating a ratio of a size of a three-dimensional image to a screen of a display of the image processing system according to the aspect of the present disclosure.

### DETAILED DESCRIPTION

Set forth below with reference to the accompanying drawings is a detailed description of embodiments of an image processing device, an image processing system, an image display method, and an image processing program. Note that since embodiments described below are preferred specific examples of the present disclosure, although various technically preferable limitations are given, the scope of the present disclosure is not limited to the embodiments unless otherwise specified in the following descriptions.

In the drawings, the same or corresponding parts are denoted by the same reference numerals. In the description of the present embodiment, the description of the same or corresponding parts will be omitted or simplified as appropriate.

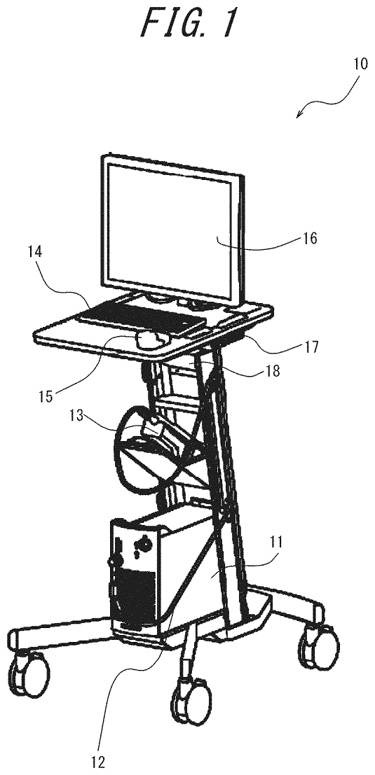

An outline of the present embodiment will be described with reference to FIGS. 1, 3, and 4A to 4C.

An image processing device 11 according to the present embodiment is a computer that causes a display 16 to display three-dimensional data 52 as a three-dimensional image 53, the three-dimensional data 52 representing a biological tissue 60. The image processing device 11 adjusts a color tone of each pixel of the three-dimensional image 53 according to a dimension of the biological tissue 60 in a linear direction from a viewpoint when the three-dimensional image 53 is displayed on the display 16.

According to the present embodiment, it is possible to facilitate understanding of a structure of at least a part of the biological tissue 60 in a visual line direction when the three-dimensional image 53 is displayed. For example, if a user is an operator, it can be relatively easy to understand a tissue structure in a direction as viewed straight from the operator, and it can be relatively easy to execute treatment on an inside of the biological tissue 60.

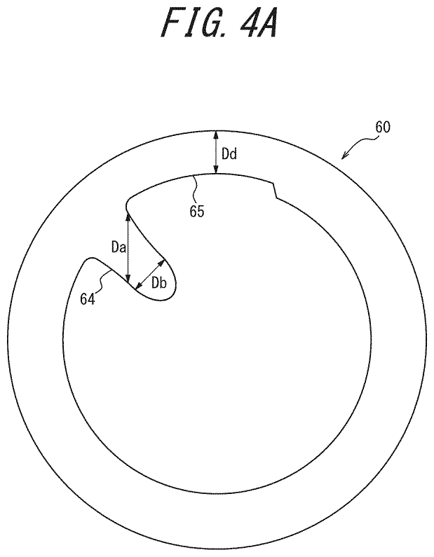

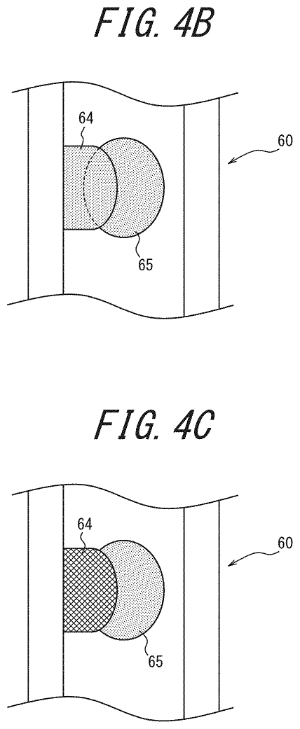

The biological tissue 60 can be, for example, an organ such as a blood vessel or a heart. In the examples of FIGS. 4A to 4C, the biological tissue 60 is a right atrium. In this example, a portion of the right atrium adjacent to a fossa ovalis 65 is raised inward to form a ridge 64. In FIGS. 4A to 4C, hatching representing a cross section of the right atrium tissue is omitted for convenience. FIGS. 4B and 4C are cross-sectional views of the biological tissue 60 of FIG. 4A viewed along a visual line direction. Here, since the visual line direction is set such that the fossa ovalis 65 can be viewed in front, a thickness direction of the fossa ovalis 65 is substantially the same as the visual line direction, but a thickness direction of the ridge 64 is different from the visual line direction. FIG. 4B illustrates, as a comparative example, an example in which a color tone change based on a dimension in the thickness direction is applied to a tissue surface. FIG. 4C illustrates an example in which a color tone change based on a dimension in the visual line direction is applied to a tissue surface in the present embodiment. In FIGS. 4B and 4C, coloring of the ridge 64 and the fossa ovalis 65 is represented by hatching for convenience.

In FIG. 4A, a dimension Db of the ridge 64 in the thickness direction is substantially the same as a dimension Dd of the fossa ovalis 65 in the thickness direction. Therefore, as illustrated in FIG. 4B, if the tissue surface is color-coded according to the dimension in the thickness direction, a boundary between the ridge 64 and the fossa ovalis 65 is almost invisible. As a result, the operator may mistakenly recognize the fossa ovalis 65 including the ridge 64, and it may be difficult to appropriately execute treatment such as atrial septal puncture. Meanwhile, a dimension Da of the ridge 64 in the visual line direction does not match the dimension Db of the ridge 64 in the thickness direction, and tends to be relatively larger than the dimension Dd of the fossa ovalis 65 in the thickness direction. Therefore, as illustrated in FIG. 4C the ridge 64 can be displayed separately from the fossa ovalis 65 by color-coding the tissue surface according to the dimension in the visual line direction. That is, it becomes relatively easy for the operator to recognize that a part of the fossa ovalis 65 is hidden in the ridge 64 by expressing a portion of the ridge 64 as it is. As a result, it can be relatively easy for the operator to understand the fossa ovalis 65 and a tissue structure around the fossa ovalis 65. Therefore, it becomes relatively easy to execute treatment such as the atrial septal puncture.

A configuration of an image processing system 10 according to the present embodiment will be described with reference to FIG. 1.

The image processing system 10 can include the image processing device 11, a cable 12, a drive unit 13, a keyboard 14, a mouse 15, and the display 16.

The image processing device 11 can be a dedicated computer specialized for image diagnosis in the present embodiment, but may also be a general-purpose computer such as a personal computer (PC).

The cable 12 is used to connect the image processing device 11 and the drive unit 13.

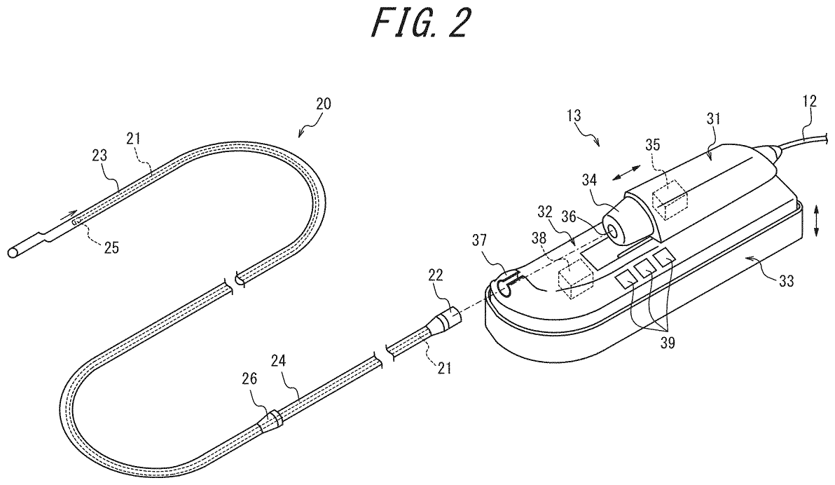

The drive unit 13 is a device to be used by connecting to a probe 20 illustrated in FIG. 2 to drive the probe 20. The drive unit 13 is also referred to as a motor drive unit (MDU). The probe 20 is applied to IVUS. The probe 20 is also referred to as an IVUS catheter or an image diagnostic catheter.

The keyboard 14, the mouse 15, and the display 16 can be connected to the image processing device 11 via any cable or wirelessly. The display 16 can be, for example, a liquid crystal display (LCD), an organic electro luminescence (EL) display, or a head-mounted display (HMD).

The image processing system 10 optionally further includes a connection terminal 17 and a cart unit 18.

The connection terminal 17 is used to connect the image processing device 11 and an external device. The connection terminal 17 can be, for example, a universal serial bus (USB) terminal. The external device can be, for example, a recording medium such as a magnetic disk drive, a magneto-optical disc drive, or an optical disc drive.

The cart unit 18 can be a cart equipped with casters for movement. The image processing device 11, the cable 12, and the drive unit 13 can be disposed on a cart body of the cart unit 18. The keyboard 14, the mouse 15, and the display 16 can be disposed on an uppermost table of the cart unit 18.

Configurations of the probe 20 and the drive unit 13 according to the present embodiment will be described with reference to FIG. 2.

The probe 20 can include a drive shaft 21, a hub 22, a sheath 23, an outer tube 24, an ultrasound transducer 25, and a relay connector 26.

The drive shaft 21 passes through the sheath 23 to be inserted into a body cavity of a living body and the outer tube 24 connected to a proximal end of the sheath 23, and extends to an inside of the hub 22 provided at a proximal end of the probe 20. The drive shaft 21 is provided with the ultrasound transducer 25, which transmits and receives signals, at a distal end of the drive shaft 21, and is rotatably provided in the sheath 23 and the outer tube 24. The relay connector 26 connects the sheath 23 and the outer tube 24.

The hub 22, the drive shaft 21, and the ultrasound transducer 25 are connected to each other so as to integrally move forward and backward in an axial direction. Therefore, for example, when the hub 22 is pressed toward a distal side, the drive shaft 21 and the ultrasound transducer 25 move inside the sheath 23 toward the distal side. For example, when the hub 22 is pulled toward a proximal side, the drive shaft 21 and the ultrasound transducer 25 move inside the sheath 23 toward the proximal side as indicated, for example, by an arrow as shown in FIG. 2.

The drive unit 13 can include a scanner unit 31, a slide unit 32, and a bottom cover 33.

The scanner unit 31 is connected to the image processing device 11 via the cable 12. The scanner unit 31 can include a probe connection section 34 connected to the probe 20, and a scanner motor 35 which is a drive source for rotating the drive shaft 21.

The probe connection section 34 can be freely detachably connected to the probe 20 through an insertion port 36 of the hub 22 provided at the proximal end of the probe 20. Inside the hub 22, a proximal end of the drive shaft 21 is rotatably supported, and a rotational force of the scanner motor 35 is transmitted to the drive shaft 21. A signal is transmitted and received between the drive shaft 21 and the image processing device 11 via the cable 12. In the image processing device 11, generation of a tomographic image of a body lumen and image processing is executed based on the signal transmitted from the drive shaft 21.

The slide unit 32 is mounted with the scanner unit 31 in a manner capable of moving forward and backward, and is mechanically and electrically connected to the scanner unit 31. The slide unit 32 includes a probe clamp section 37, a slide motor 38, and a switch group 39.

The probe clamp section 37 is disposed coaxially with the probe connection section 34 on the distal side relative to the probe connection section 34, and supports the probe 20 to be connected to the probe connection section 34.

The slide motor 38 is a drive source that generates a driving force in the axial direction. The scanner unit 31 moves forward and backward when driven by the slide motor 38, and the drive shaft 21 moves forward and backward in the axial direction accordingly. The slide motor 38 can be, for example, a servo motor.

The switch group 39 can include, for example, a forward switch and a pull-back switch that are pressed when the scanner unit 31 is to be moved forward or backward, and a scan switch that is pressed when image drawing is to be started or ended. Various switches may be included in the switch group 39 as necessary without being limited to the example here.

When the forward switch is pressed, the slide motor 38 rotates forward, and the scanner unit 31 moves forward. Meanwhile, when the pull-back switch is pressed, the slide motor 38 rotates backward, and the scanner unit 31 moves backward.

When the scan switch is pressed, the image drawing is started, the scanner motor 35 is driven, and the slide motor 38 is driven to move the scanner unit 31 backward. The user such as the operator connects the probe 20 to the scanner unit 31 in advance, such that the drive shaft 21 rotates and moves toward the proximal side in the axial direction upon the start of the image drawing. When the scan switch is pressed again, the scanner motor 35 and the slide motor 38 are stopped, and the image drawing is ended.

The bottom cover 33 covers a bottom and an entire circumference of a side surface on a bottom side of the slide unit 32, and is capable of moving toward and away from the bottom of the slide unit 32.

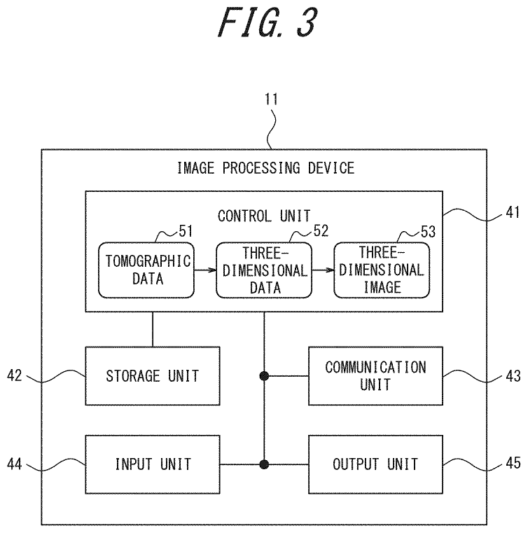

A configuration of the image processing device 11 will be described with reference to FIG. 3.

The image processing device 11 can include a control unit 41, a storage unit 42, a communication unit 43, an input unit 44, and an output unit 45.

The control unit 41 includes at least one processor, at least one dedicated circuit, or a combination of the at least one processor and the at least one dedicated circuit. The processor can be a general-purpose processor such as a central processing unit (CPU) or a graphics processing unit (GPU), or a dedicated processor specialized for specific processing. The dedicated circuit can be, for example, a field-programmable gate array (FPGA) or an application specific integrated circuit (ASIC). The control unit 41 executes processing related to an operation of the image processing device 11 while controlling each unit of the image processing system 10 including the image processing device 11.

The storage unit 42 can include at least one semiconductor memory, at least one magnetic memory, at least one optical memory, or a combination of at least two of the at least one semiconductor memory, the at least one magnetic memory, and the at least one optical memory. The semiconductor memory can be, for example, a random-access memory (RAM) or a read only memory (ROM). The RAM can be, for example, a static random-access memory (SRAM) or a dynamic random-access memory (DRAM). The ROM can be, for example, an electrically erasable programmable read only memory (EEPROM). The storage unit 42 can function as, for example, a main storage device, an auxiliary storage device, or a cache memory. The storage unit 42 stores data used for the operation of the image processing device 11, such as tomographic data 51, and data obtained by the operation of the image processing device 11, such as the three-dimensional data 52 and the three-dimensional image 53.

The communication unit 43 includes at least one communication interface. The communication interface can be, for example, a wired local area network (LAN) interface, a wireless LAN interface, or an image diagnostic interface for receiving IVUS signals and executing analog to digital (A/D) conversion for the IVUS signals. The communication unit 43 receives the data used for the operation of the image processing device 11 and transmits the data obtained by the operation of the image processing device 11. In the present embodiment, the drive unit 13 is connected to the image diagnostic interface included in the communication unit 43.

The input unit 44 includes at least one input interface. The input interface can be, for example, a USB interface, a High-Definition Multimedia Interface (HDMI®) interface, or an interface compatible with short-range wireless communication such as Bluetooth®. The input unit 44 receives an operation of the user such as an operation of inputting the data used for the operation of the image processing device 11. In the present embodiment, the keyboard 14 and the mouse 15 are connected to the USB interface or the interface compatible with short-range wireless communication included in the input unit 44. When a touch screen is provided integrally with the display 16, the display 16 may be connected to the USB interface or the HDMI interface included in the input unit 44.

The output unit 45 includes at least one output interface. The output interface can be, for example, a USB interface, an HDMI interface, or an interface compatible with short-range wireless communication such as Bluetooth. The output unit 45 outputs the data obtained by the operation of the image processing device 11. In the present embodiment, the display 16 is connected to the USB interface or the HDMI interface included in the output unit 45.

A function of the image processing device 11 is implemented by executing an image processing program according to the present embodiment by the processor corresponding to the control unit 41. That is, the function of the image processing device 11 is implemented by software. The image processing program causes a computer to function as the image processing device 11 by causing the computer to execute processing of the image processing device 11. That is, the computer functions as the image processing device 11 by executing the processing of the image processing device 11 according to the image processing program.

The program may be stored in a non-transitory computer-readable medium in advance. The non-transitory computer-readable medium is, for example, a flash memory, a magnetic recording device, an optical disc, a magneto-optical recording medium, or a ROM. Distribution of the program is executed by, for example, selling, transferring, or lending a portable medium such as a secure digital (SD) card, a digital versatile disc (DVD) or a compact disc read only memory (CD-ROM) storing the program. The program may be distributed by storing the program in a storage of a server in advance and transferring the program from the server to another computer. The program may be provided as a program product.

For example, the computer temporarily stores, in the main storage device, the program stored in the portable medium or the program transferred from the server. The computer reads, by the processor, the program stored in the main storage device, and executes, by the processor, processing according to the read program. The computer may read the program directly from the portable medium and execute the processing according to the program. Each time the program is transferred from the server to the computer, the computer may sequentially execute processing according to the received program. The processing may be executed by a so-called application service provider (ASP) type service in which the function is implemented only by execution instruction and result acquisition without transferring the program from the server to the computer. The program includes information provided for processing by an electronic computer and conforming to the program. For example, data that is not a direct command to the computer but has a property that defines the processing of the computer corresponds to the “information conforming to the program”.

The functions of the image processing device 11 may be partially or entirely implemented by the dedicated circuit corresponding to the control unit 41. That is, the functions of the image processing device 11 may be partially or entirely implemented by hardware.

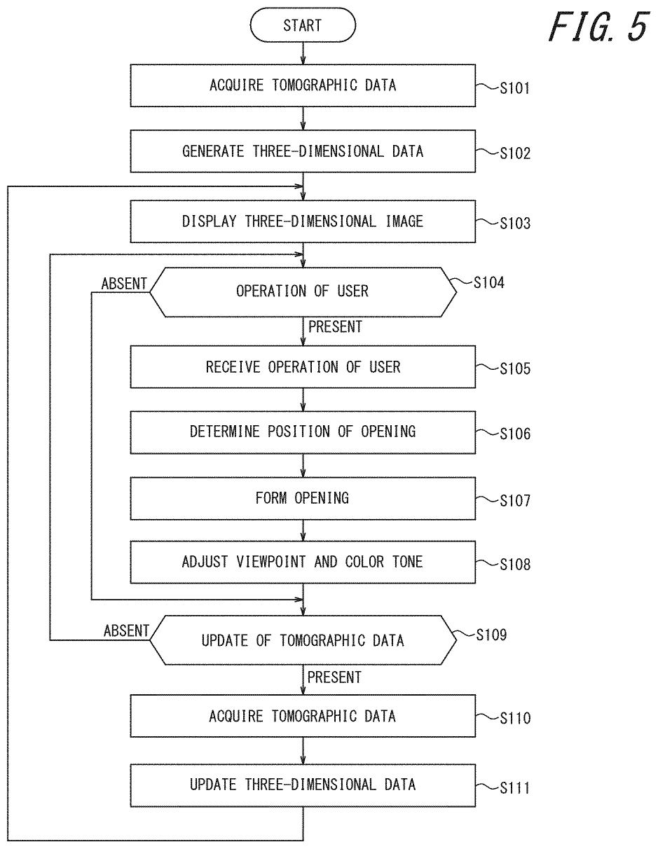

An operation of the image processing system 10 according to the present embodiment will be described with reference to FIG. 5. The operation of the image processing system 10 corresponds to an image display method according to the present embodiment.

Before a start of a flow in FIG. 5, the probe 20 is primed by the user. Thereafter, the probe 20 is fitted into the probe connection section 34 and the probe clamp section 37 of the drive unit 13, and is connected and fixed to the drive unit 13. Then, the probe 20 is inserted to a target site in the biological tissue 60 such as the blood vessel or the heart.

In S101, the scan switch included in the switch group 39 is pressed, and a so-called pull-back operation is executed by pressing the pull-back switch included in the switch group 39. The probe 20 transmits an ultrasound wave inside the biological tissue 60 by the ultrasound transducer 25 that moves backward in the axial direction by the pull-back operation. The ultrasound transducer 25 radially transmits the ultrasound wave while moving inside the biological tissue 60. The ultrasound transducer 25 receives a reflected wave of the transmitted ultrasound wave. The probe 20 inputs a signal of the reflected wave received by the ultrasound transducer 25 to the image processing device 11. The control unit 41 of the image processing device 11 processes the input signal to sequentially generate cross-sectional images of the biological tissue 60, thereby acquiring the tomographic data 51, which includes a plurality of cross-sectional images.

Specifically, the probe 20 transmits the ultrasound wave in a plurality of directions from a rotation center to an outside by the ultrasound transducer 25 while causing the ultrasound transducer 25 to rotate in a circumferential direction and to move in the axial direction inside the biological tissue 60. The probe 20 receives the reflected wave from a reflecting object present in each of the plurality of directions inside the biological tissue 60 by the ultrasound transducer 25. The probe 20 transmits the signal of the received reflected wave to the image processing device 11 via the drive unit 13 and the cable 12. The communication unit 43 of the image processing device 11 receives the signal transmitted from the probe 20. The communication unit 43 executes A/D conversion for the received signal. The communication unit 43 inputs the A/D-converted signal to the control unit 41. The control unit 41 processes the input signal to calculate an intensity value distribution of the reflected wave from the reflecting object present in a transmission direction of the ultrasound wave of the ultrasound transducer 25. The control unit 41 sequentially generates two-dimensional images having a luminance value distribution corresponding to the calculated intensity value distribution as the cross-sectional images of the biological tissue 60, thereby acquiring the tomographic data 51 which is a data set of the cross-sectional images. The control unit 41 stores the acquired tomographic data 51 in the storage unit 42.

In the present embodiment, the signal of the reflected wave received by the ultrasound transducer 25 corresponds to raw data of the tomographic data 51, and the cross-sectional images generated by processing the signal of the reflected wave by the image processing device 11 correspond to processed data of the tomographic data 51.

In a modification of the present embodiment, the control unit 41 of the image processing device 11 may store the signal input from the probe 20 as it is in the storage unit 42 as the tomographic data 51. Alternatively, the control unit 41 may store data indicating the intensity value distribution of the reflected wave calculated by processing the signal input from the probe 20 in the storage unit 42 as the tomographic data 51. That is, the tomographic data 51 is not limited to the data set of the cross-sectional images of the biological tissue 60, and may be data representing a cross section of the biological tissue 60 at each moving position of the ultrasound transducer 25 in any format.

In a modification of the present embodiment, an ultrasound transducer that transmits the ultrasound wave in the plurality of directions without rotating may be used instead of the ultrasound transducer 25 that transmits the ultrasound wave in the plurality of directions while rotating in the circumferential direction.

In a modification of the present embodiment, the tomographic data 51 may be acquired using optical frequency domain imaging (OFDI) or optical coherence tomography (OCT) instead of being acquired by using IVUS. When OFDI or OCT is used, as a sensor that acquires the tomographic data 51 while moving in the lumen of the biological tissue 60, a sensor that acquires the tomographic data 51 by emitting light in the lumen of the biological tissue 60 is used instead of the ultrasound transducer 25 that acquires the tomographic data 51 by transmitting the ultrasound wave in the lumen of the biological tissue 60.

In a modification of the present embodiment, instead of the image processing device 11 generating the data set of the cross-sectional images of the biological tissue 60, another device may generate the same data set, and the image processing device 11 may acquire the data set from the other device. That is, instead of the control unit 41 of the image processing device 11 processing the IVUS signal to generate the cross-sectional images of the biological tissue 60, another device may process the IVUS signal to generate the cross-sectional images of the biological tissue 60 and input the generated cross-sectional images to the image processing device 11.

In S102, the control unit 41 of the image processing device 11 generates the three-dimensional data 52 of the biological tissue 60 based on the tomographic data 51 acquired in S101.

Specifically, the control unit 41 of the image processing device 11 generates the three-dimensional data 52 of the biological tissue 60 by stacking the cross-sectional images of the biological tissue 60 included in the tomographic data 51 stored in the storage unit 42, and converting the same into three-dimensional data. As a method for three-dimensional conversion, any method among a rendering method such as surface rendering or volume rendering, and various processing such as texture mapping including environment mapping, and bump mapping, which is associated with the rendering method, is used. The control unit 41 stores the generated three-dimensional data 52 in the storage unit 42.

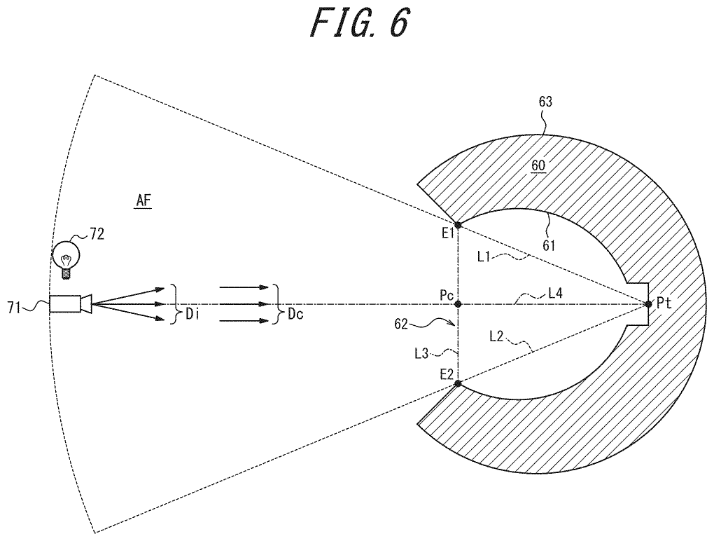

In S103, the control unit 41 of the image processing device 11 displays the three-dimensional data 52 generated in S102 on the display 16 as the three-dimensional image 53. At this time point, the control unit 41 may arrange, at any positions, the viewpoint and a virtual light source 72 when displaying the three-dimensional image 53 on the display 16. The term “viewpoint” refers to a position of a virtual camera 71 disposed in a three-dimensional space, as illustrated in FIG. 6. The number and relative position of the light source 72 are not limited to those illustrated in the figure, and can be changed as appropriate.

Specifically, the control unit 41 of the image processing device 11 generates the three-dimensional image 53 based on the three-dimensional data 52 stored in the storage unit 42. The control unit 41 displays the generated three-dimensional image 53 on the display 16 via the output unit 45.

In S104, if there is an operation of the user, processing of S105 to S108 is executed. If there is no operation of the user, the processing of S105 to S108 can be skipped.

In S105, the control unit 41 of the image processing device 11 receives, via the input unit 44, an operation of setting a position of an opening 62 as illustrated in FIG. 6. The position of the opening 62 is set to a position at which the lumen of the biological tissue 60 is exposed through the opening 62 in the three-dimensional image 53 displayed in S103.

Specifically, the control unit 41 of the image processing device 11 receives, via the input unit 44, an operation of the user cutting off a portion of the biological tissue 60 using the keyboard 14, the mouse 15, or the touch screen provided integrally with the display 16 in the three-dimensional image 53 displayed on the display 16. In the example of FIG. 6, the control unit 41 receives an operation of cutting off a portion of the biological tissue 60 such that an inner surface 61 of the biological tissue 60 has an opened shape in the cross section of the biological tissue 60. The term “cross section of the biological tissue 60” refers to, for example, a tomographic cross section having two end edges of the opening 62 facing each other and the inner surface 61 of the biological tissue 60 facing the opening 62, but is not limited to this tomographic cross section, and may be a transverse cross section of the biological tissue 60, a vertical cross section of the biological tissue 60, or another cross section of the biological tissue 60. The term “transverse cross section of the biological tissue 60” refers to a cross section obtained by cutting the biological tissue 60 perpendicularly to a direction in which the ultrasound transducer 25 moves in the biological tissue 60. The term “vertical cross section of the biological tissue 60” refers to a cross section obtained by cutting the biological tissue 60 along the direction in which the ultrasound transducer 25 moves in the biological tissue 60. The term “another cross section of the biological tissue 60” refers to a cross section obtained by cutting the biological tissue 60 obliquely with respect to the direction in which the ultrasound transducer 25 moves in the biological tissue 60. The term “opened shape” refers to, for example, a substantially C shape, a substantially U shape, a substantially “3” shape, or a shape partially missing due to the presence of a hole originally free in the biological tissue 60, such as a bifurcated portion of the blood vessel or a pulmonary vein ostia. In the example of FIG. 6, a shape of the inner surface 61 of the biological tissue 60 is a substantially C shape, and a portion facing the opening 62 is missing.

In S106, the control unit 41 of the image processing device 11 determines the position set by the operation received in S105 as the position of the opening 62.

Specifically, the control unit 41 of the image processing device 11 specifies, as three-dimensional coordinates of an edge of the opening 62, three-dimensional coordinates of a boundary of a portion of the biological tissue 60 cut by the operation of the user in the three-dimensional data 52 stored in the storage unit 42. The control unit 41 stores the specified three-dimensional coordinates in the storage unit 42.

In S107, the control unit 41 of the image processing device 11 forms, in the three-dimensional data 52, the opening 62 exposing the lumen of the biological tissue 60 in the three-dimensional image 53.

Specifically, the control unit 41 of the image processing device 11 hides a portion in the three-dimensional data 52 stored in the storage unit 42 that is specified by the three-dimensional coordinates stored in the storage unit 42 or sets the portion to be transparent when the three-dimensional image 53 is to be displayed on the display 16.

In S108, the control unit 41 of the image processing device 11 adjusts the viewpoint when displaying the three-dimensional image 53 on the display 16 according to the position of the opening 62 formed in S107. In the present embodiment, the control unit 41 arranges the viewpoint on a straight line extending from the inner surface 61 of the biological tissue 60 to an outside of the biological tissue 60 through the opening 62. Accordingly, the user can virtually observe the inner surface 61 of the biological tissue 60 by looking into the biological tissue 60 through the opening 62.

Specifically, the control unit 41 of the image processing device 11 arranges the virtual camera 71 at a position at which the inner surface 61 of the biological tissue 60 can be viewed through the portion hidden or set to be transparent in the three-dimensional image 53 displayed on the display 16. In the example of FIG. 6, the control unit 41 arranges the virtual camera 71 in a region AF interposed between a first straight line L1 and a second straight line L2 in the cross section of the biological tissue 60. The first straight line L1 extends from the inner surface 61 of the biological tissue 60 to the outside of the biological tissue 60 through a first end edge E1 of the opening 62. The second straight line L2 extends from the inner surface 61 of the biological tissue 60 to the outside of the biological tissue 60 through a second end edge E2 of the opening 62. A point at which the first straight line L1 intersects the inner surface 61 of the biological tissue 60 is a point Pt identical to a point at which the second straight line L2 intersects the inner surface 61 of the biological tissue 60. Accordingly, the user can observe the point Pt on the inner surface 61 of the biological tissue 60 regardless of a position of the virtual camera 71 in the region AF.

In the example of FIG. 6, the point Pt is identical to a point at which a fourth straight line L4 intersects the inner surface 61 of the biological tissue 60. The fourth straight line L4 is drawn perpendicularly to a third straight line L3 from a midpoint Pc of the third straight line L3. The third straight line L3 connects the first end edge E1 of the opening 62 and the second end edge E2 of the opening 62. Accordingly, the user can rather easily observe the point Pt on the inner surface 61 of the biological tissue 60 through the opening 62. In particular, as illustrated in FIG. 6, when the virtual camera 71 is arranged on an extension line of the fourth straight line L4, the user can rather easily observe the point Pt on the inner surface 61 of the biological tissue 60.

The position of the virtual camera 71 may be any position at which the inner surface 61 of the biological tissue 60 can be observed through the opening 62, and is within a range facing the opening 62 in the present embodiment. The position of the virtual camera 71 can be preferably set to an intermediate position facing a central portion of the opening 62.

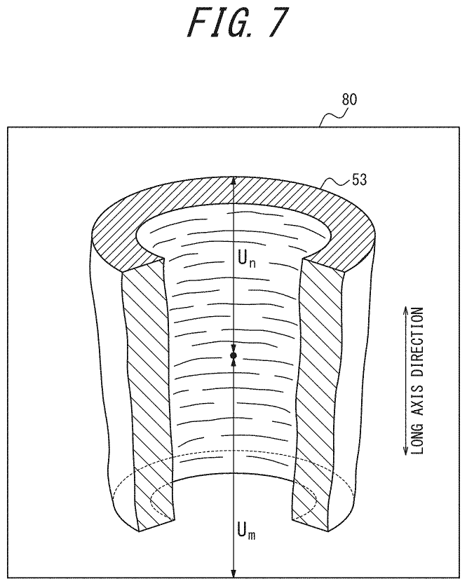

In the example of FIG. 7, a minimum value Smin and a maximum value Smax are set for a ratio S of a distance Un from a center to one end of the three-dimensional image 53 displayed on a screen 80 of the display 16 to a distance Um from a center to one end of the screen 80 such that the centers of the screen 80 and the three-dimensional image 53 overlap with each other. For example, Smin is set to ⅓, and Smax is set to 1. In the example of FIG. 6, a minimum distance from the point Pt to the position of the camera 71 may be set according to the minimum value Smin, and a maximum distance from the point Pt to the position of the virtual camera 71 may be set according to the maximum value Smax. Alternatively, the minimum distance from the point Pt to the position of the camera 71 may be set to such a distance that the camera 71 is not closer to the point Pt than the opening 62 regardless of the minimum value Smin. The maximum distance from the point Pt to the position of the virtual camera 71 may be set to such a distance that the camera 71 is not farther from the point Pt more than such a distance that the user cannot observe the inner surface 61 of the biological tissue 60 regardless of the maximum value Smax.

Further, in S108, the control unit 41 of the image processing device 11 adjusts the color tone of each pixel of the three-dimensional image 53 according to the dimension of the biological tissue 60 in the linear direction from the viewpoint when the three-dimensional image 53 is displayed on the display 16. The linear direction from the viewpoint may be a direction common to all the pixels, or may be different directions depending on the pixel. In the former case, a direction in which any one pixel of the three-dimensional image 53 is viewed straight from the viewpoint is set to be common to all the pixels including the one pixel. In the latter case, a direction in which each pixel of the three-dimensional image 53 is viewed straight from the viewpoint can be individually set for each pixel.

Specifically, the control unit 41 of the image processing device 11 sets, as a common visual line direction Dc as illustrated in FIG. 6, a linear direction from the viewpoint toward one point on the inner surface 61 of the biological tissue 60 in the three-dimensional data 52. The control unit 41 calculates, as a dimension of the biological tissue 60 in the visual line direction Dc, a distance from each point on the inner surface 61 of the biological tissue 60 to a corresponding point in the visual line direction Dc on an outer surface 63 of the biological tissue 60. Alternatively, the control unit 41 can set, as an individual visual line direction Di as illustrated in FIG. 6, a linear direction from the viewpoint toward each point on the inner surface 61 of the biological tissue 60 in the three-dimensional data 52. The control unit 41 calculates, as a dimension of the biological tissue 60 in the visual line direction Di, a distance from each point on the inner surface 61 of the biological tissue 60 to a corresponding point in the visual line direction Di on the outer surface 63 of the biological tissue 60. Then, the control unit 41 stores the calculated distance in the storage unit 42 for each point on the inner surface 61. The control unit 41 converts, for each point on the inner surface 61, the distance stored in the storage unit 42 into the color tone by using a conversion formula or a conversion table set in advance. The control unit 41 stores, in the storage unit 42, the color tone calculated using the conversion formula or the conversion table for each point on the inner surface 61. The control unit 41 sets the color tone of the corresponding pixel of the three-dimensional image 53 to the color tone stored in the storage unit 42 for each point on the inner surface 61. As a method for setting the color tone of each pixel, any method such as a method of calculating an RGB value (i.e., red, green, blue value), or a method of calculating an ARGB value (Addressable RGB value) including transparency in the RGB value may be used.

In S109, if the tomographic data 51 is updated, processing of S110 and S111 is executed. If the tomographic data 51 is not updated, the presence or absence of the operation of the user is confirmed again in S104.

In S110, similarly to the processing of S101, the control unit 41 of the image processing device 11 processes the signal input from the probe 20 to newly generate cross-sectional images of the biological tissue 60, thereby acquiring the tomographic data 51 including at least one new cross-sectional image.

In S111, the control unit 41 of the image processing device 11 updates the three-dimensional data 52 of the biological tissue 60 based on the tomographic data 51 acquired in S110. Then, in S103, the control unit 41 displays the three-dimensional data 52 updated in S111 on the display 16 as the three-dimensional image 53.

In S105 to S108 for the second and subsequent times, when the position of the opening 62 is changed from a first position to a second position, the control unit 41 of the image processing device 11 moves the viewpoint from a third position corresponding to the first position to a fourth position corresponding to the second position. The control unit 41 moves the virtual light source 72 when the three-dimensional image 53 is to be displayed on the display 16, in accordance with the movement of the viewpoint from the third position to the fourth position.

The control unit 41 moves the virtual light source 72 by using a rotation matrix used for moving the virtual camera 71 when changing a position of the opening 62 in the circumferential direction in the cross section of the biological tissue 60.

The control unit 41 may instantaneously switch the viewpoint from the third position to the fourth position when changing the position of the opening 62 from the first position to the second position, but in the present embodiment, a video in which the viewpoint gradually moves from the third position to the fourth position is displayed on the display 16 as the three-dimensional image 53. Therefore, the movement of the viewpoint is rather easily introduced to the user.

In a modification of the present embodiment, in S105, the control unit 41 of the image processing device 11 may receive, via the input unit 44, an operation of setting a position of a target point that the user wants to view and the operation of setting the position of the opening 62.

Specifically, in the three-dimensional image 53 displayed on the display 16, the control unit 41 of the image processing device 11 may receive, via the input unit 44, the operation by the user of designating the position of the target point using the keyboard 14, the mouse 15, or the touch screen provided integrally with the display 16. In the example of FIG. 6, the control unit 41 may receive, via the input unit 44, an operation of setting a position of the point Pt as the position of the point at which the first straight line L1 and the second straight line L2 intersect the inner surface 61 of the biological tissue 60.

In a modification of the present embodiment, in S105, the control unit 41 of the image processing device 11 may receive, via the input unit 44, the operation of setting the position of the target point that the user wants to view, instead of the operation of setting the position of the opening 62. Then, in S106, the control unit 41 may determine the position of the opening 62 according to the position set by the operation received in S105.

Specifically, the control unit 41 of the image processing device 11 may receive, via the input unit 44, the operation by the user of designating the position of the target point in the three-dimensional image 53 displayed on the display 16 using the keyboard 14, the mouse 15, or the touch screen provided integrally with the display 16. Then, the control unit 41 may determine the position of the opening 62 according to the position of the target point. In the example of FIG. 6, the control unit 41 may receive, via the input unit 44, the operation of setting the position of the point Pt as the position of the point at which the first straight line L1 and the second straight line L2 intersect the inner surface 61 of the biological tissue 60. In the cross section of the biological tissue 60, the control unit 41 may determine, as the region AF, a fan-shaped region centered on the point Pt and having a central angle that is preset or an angle that is specified by the user. The control unit 41 may determine a position in the biological tissue 60 that overlaps with the region AF as the position of the opening 62. The control unit 41 may determine a normal line of the inner surface 61 of the biological tissue 60, which is perpendicular to a tangent line passing through the point Pt, as the fourth straight line L4.

The region AF may be set to be narrower than a width of the opening 62. That is, the region AF may be set so as not to include at least one of the first end edge E1 of the opening 62 and the second end edge E2 of the opening 62.

In a modification of the present embodiment, the point at which the first straight line L1 intersects the inner surface 61 of the biological tissue 60 may not be identical to the point at which the second straight line L2 intersects the inner surface 61 of the biological tissue 60. For example, a point P1 at which the first straight line L1 intersects the inner surface 61 of the biological tissue 60 and a point P2 at which the second straight line L2 intersects the inner surface 61 of the biological tissue 60 may be on a circumference centered on the point Pt. That is, the point P1 and the point P2 may be substantially equidistant from the point Pt.

In a modification of the present embodiment, the control unit 41 of the image processing device 11 may set a color of a pixel in which the dimension of the biological tissue 60 in the linear direction from the viewpoint is smaller than a first threshold value to a first color different from those of the other pixels. The “first color” can be, for example, red, but any color may be used as long as the pixel to be colored can be distinguished from the other pixels. For example, by setting the first threshold value between 0.5 mm and 8.0 mm, the portion of the fossa ovalis 65 can be made conspicuous as illustrated in FIG. 4C. Note that the coloring of the ridge 64 is represented by hatching in FIG. 4C, the color of the ridge 64 may be the same as a part other than the fossa ovalis 65, such as a tissue surface around the fossa ovalis 65. For example, the fossa ovalis 65 may be colored red, and the ridge 64 and the tissue surface around the fossa ovalis 65 may be colored blue. Note that the first threshold value can be, for example, preferably set between 1.0 mm and 5.0 mm.

In a modification of the present embodiment, the control unit 41 of the image processing device 11 may switch the display mode between the first mode of adjusting the color tone of each pixel of the three-dimensional image 53 according to the dimension of the biological tissue 60 in the linear direction from the viewpoint, and the second mode of adjusting the color tone of each pixel of the three-dimensional image 53 according to the dimension of the biological tissue 60 in the thickness direction of the biological tissue 60. According to this modification, it is relatively easy for the operator to understand the fossa ovalis 65 and the tissue structure around the fossa ovalis 65 at the time of the atrial septal puncture, and it is relatively easy for the operator to recognize a tissue thickness of a target portion of ablation during cardiac ablation for treating arrhythmia.

In the first mode, as described above, the control unit 41 of the image processing device 11 may set a color of a pixel among a pixel group of the three-dimensional image 53 in which the dimension of the biological tissue 60 in the linear direction from the viewpoint is smaller than the first threshold value to the first color different from those of the other pixels.

In the second mode, the control unit 41 of the image processing device 11 may set a color of a pixel among the pixel group of the three-dimensional image 53 in which the dimension of the biological tissue 60 in the thickness direction of the biological tissue 60 is larger than a second threshold value to a second color different from those of the other pixels. The “second color” is, for example, blue, but any color may be used as long as the pixel to be colored can be distinguished from other pixels. For example, by setting the threshold value to 1.0 mm or more, in the cardiac ablation for treating arrhythmia, a portion having a larger tissue thickness to which a larger energy than usual should be given can be made conspicuous.

Alternatively, in the second mode, among the pixel group of the three-dimensional image 53, the control unit 41 of the image processing device 11 sets a color of a pixel in which the dimension of the biological tissue 60 in the thickness direction of the biological tissue 60 is larger than the second threshold value to the second color, sets a color of a pixel in which the dimension of the biological tissue 60 in the thickness direction of the biological tissue 60 is smaller than a third threshold value that is less than the second threshold value to a third color different from the second color, and sets a color of a pixel in which the dimension of the biological tissue 60 in the thickness direction of the biological tissue 60 is equal to or greater than the third threshold value and equal to or smaller than the second threshold value to a color different from the second color and the third color. For example, by setting the second threshold value to 1.0 mm and the third threshold value to 0.5 mm, in the cardiac ablation for treating arrhythmia, both a portion having a small tissue thickness that should be taken care of so that the tissue is not broken through when pressing the ablation catheter, and a portion having a large tissue thickness that should be given a larger energy than usual can be made conspicuous.

The switching of the display mode may be manually executed by the operation of the user, or may be automatically executed by using any event as a trigger.

As described above, in the present embodiment, the control unit 41 of the image processing device 11 causes the display 16 to display the three-dimensional data 52 as the three-dimensional image 53, the three-dimensional data 52 representing the biological tissue 60. The control unit 41 adjusts the color tone of each pixel of the three-dimensional image 53 according to the dimension of the biological tissue 60 in the linear direction from the viewpoint when the three-dimensional image 53 is displayed on the display 16.

According to the present embodiment, it is possible to facilitate understanding of a structure of at least a part of the biological tissue 60 in the visual line direction when the three-dimensional image 53 is displayed. For example, if the user is an operator, it is relatively easy to understand the tissue structure in the direction as viewed straight from the operator, and it is relatively easy to execute treatment on the inside of the biological tissue 60.

In the present embodiment, once the position of the opening 62 is determined, the positions of the camera 71 and the light source 72 are moved such that the inside of the biological tissue 60 can be viewed from the opening 62. Therefore, it is possible to avoid a situation where only the outer surface 63 of the biological tissue 60 can be viewed and an object of interest cannot be confirmed when the position of the opening 62 is changed to another position.

The present disclosure is not limited to the above embodiments. For example, a plurality of blocks described in the block diagram may be integrated, or one block may be divided. Instead of executing a plurality of steps described in the flowchart in time series according to the description, the steps may be executed in parallel or in a different order according to the processing capability of the device that executes each step or as necessary. In addition, modifications can be made without departing from a gist of the present disclosure.

The detailed description above describes embodiments of an image processing device, an image processing system, an image display method, and an image processing program. These disclosed embodiments represent examples of the image processing device, the image processing system, the image display method, and the image processing program. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. An image processing device configured to cause a display to display three-dimensional data as a three-dimensional image, the three-dimensional data representing a biological tissue, the image processing device comprising:
a control unit configured to adjust a color tone of each pixel of the three-dimensional image according to a dimension of the biological tissue in a linear direction from a viewpoint when the three-dimensional image is displayed on the display.

2. The image processing device according to claim 1, wherein the control unit is configured to set a color of a pixel among a pixel group of the three-dimensional image in which the dimension of the biological tissue in the linear direction is smaller than a first threshold value to a first color different from those of the other pixels.

3. The image processing device according to claim 1, wherein the control unit is configured to switch a display mode between a first mode of adjusting the color tone of each pixel of the three-dimensional image according to the dimension of the biological tissue in the linear direction, and a second mode of adjusting the color tone of each pixel of the three-dimensional image according to a dimension of the biological tissue in a thickness direction of the biological tissue.

4. The image processing device according to claim 3, wherein in the second mode, the control unit is configured to set a color of a pixel among a pixel group of the three-dimensional image in which the dimension of the biological tissue in the thickness direction of the biological tissue is larger than a second threshold value to a second color different from those of the other pixels.

5. The image processing device according to claim 3, wherein in the second mode, among a pixel group of the three-dimensional image, the control unit configured to set a color of a pixel in which the dimension of the biological tissue in the thickness direction of the biological tissue is larger than a second threshold value to a second color, to set a color of a pixel in which the dimension of the biological tissue in the thickness direction of the biological tissue is smaller than a third threshold value that is less than the second threshold value to a third color different from the second color, and to set a color of a pixel in which the dimension of the biological tissue in the thickness direction of the biological tissue is equal to or greater than the third threshold value and equal to or smaller than the second threshold value to a color different from the second color and the third color.

6. The image processing device according to claim 1, wherein the control unit is configured to form, in the three-dimensional data, an opening for exposing a lumen of the biological tissue in the three-dimensional image, and to adjust a position of the viewpoint according to a position of the formed opening.

7. An image processing system, comprising:
a sensor configured to acquire tomographic data of the biological tissue while moving in a lumen of the biological tissue; and
the image processing device according to claim 1, the image processing device configured to generate the three-dimensional data based on the tomographic data acquired by the sensor.

8. The image processing system according to claim 7, further comprising:
the display.

9. The image processing system according to claim 7, wherein the control unit is configured to set a color of a pixel among a pixel group of the three-dimensional image in which the dimension of the biological tissue in the linear direction is smaller than a first threshold value to a first color different from those of the other pixels.

10. The image processing system according to claim 7, wherein the control unit is configured to switch a display mode between a first mode of adjusting the color tone of each pixel of the three-dimensional image according to the dimension of the biological tissue in the linear direction, and a second mode of adjusting the color tone of each pixel of the three-dimensional image according to a dimension of the biological tissue in a thickness direction of the biological tissue.

11. The image processing system according to claim 10, wherein in the second mode, the control unit is configured to set a color of a pixel among a pixel group of the three-dimensional image in which the dimension of the biological tissue in the thickness direction of the biological tissue is larger than a second threshold value to a second color different from those of the other pixels.

12. The image processing system according to claim 10, wherein in the second mode, among a pixel group of the three-dimensional image, the control unit configured to set a color of a pixel in which the dimension of the biological tissue in the thickness direction of the biological tissue is larger than a second threshold value to a second color, to set a color of a pixel in which the dimension of the biological tissue in the thickness direction of the biological tissue is smaller than a third threshold value that is less than the second threshold value to a third color different from the second color, and to set a color of a pixel in which the dimension of the biological tissue in the thickness direction of the biological tissue is equal to or greater than the third threshold value and equal to or smaller than the second threshold value to a color different from the second color and the third color.

13. The image processing system according to claim 7, wherein the control unit is configured to form, in the three-dimensional data, an opening for exposing a lumen of the biological tissue in the three-dimensional image, and to adjust a position of the viewpoint according to a position of the formed opening.

14. An image display method for causing a display to display three-dimensional data as a three-dimensional image, the three-dimensional data representing a biological tissue, the image display method comprising:
adjusting, by a processor, a color tone of each pixel of the three-dimensional image according to a dimension of the biological tissue in a linear direction from a viewpoint when the three-dimensional image is displayed on the display.

15. The image display method according to claim 14, further comprising:
setting, by the processor, a color of a pixel among a pixel group of the three-dimensional image in which the dimension of the biological tissue in the linear direction is smaller than a first threshold value to a first color different from those of the other pixels.

16. The image display method according to claim 14, further comprising:
switching, by the processor, a display mode between a first mode of adjusting the color tone of each pixel of the three-dimensional image according to the dimension of the biological tissue in the linear direction, and a second mode of adjusting the color tone of each pixel of the three-dimensional image according to a dimension of the biological tissue in a thickness direction of the biological tissue.

17. The image display method according to claim 16, further comprising:
Setting, by the processor, a color of a pixel among a pixel group of the three-dimensional image in which the dimension of the biological tissue in the thickness direction of the biological tissue is larger than a second threshold value to a second color different from those of the other pixels.

18. The image processing method according to claim 16, wherein in the second mode, among a pixel group of the three-dimensional image, further comprising:
setting, by the processor, a color of a pixel in which the dimension of the biological tissue in the thickness direction of the biological tissue is larger than a second threshold value to a second color;
setting a color of a pixel in which the dimension of the biological tissue in the thickness direction of the biological tissue is smaller than a third threshold value that is less than the second threshold value to a third color different from the second color; and
setting a color of a pixel in which the dimension of the biological tissue in the thickness direction of the biological tissue is equal to or greater than the third threshold value and equal to or smaller than the second threshold value to a color different from the second color and the third color.

19. The image processing method according to claim 14, further comprising:
forming in the three-dimensional data, by the processor, an opening for exposing a lumen of the biological tissue in the three-dimensional image, and to adjust a position of the viewpoint according to a position of the formed opening.

20. A non-transitory computer-readable medium (CRM) storing computer program code executed by a computer processor that executes an imaging process comprising:
displaying three-dimensional data as a three-dimensional image on a display, the three-dimensional data representing a biological tissue; and
adjusting a color tone of each pixel of the three-dimensional image according to a dimension of the biological tissue in a linear direction from a viewpoint when the three-dimensional image is displayed on the display.
